(19) United States
(12) Patent Application Publication
(10) Pub. No.: US 2005/0075571 A1
(43) Pub. Date: Apr. 7, 2005

(54) Sound absorption backings for ultrasound transducers

(75) Inventor: Stephen R. Barnes, Bellevue, WA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/665,334

(22) Filed: Sep. 18, 2003

(51) Int. Cl. A61B 8/14

(52) U.S. Cl. 600/459

(57) ABSTRACT

Sound absorption backings for ultrasound transducers are provided. A block of material with similar acoustic impedance to the transducer material is provided adjacent to the material. For example, a solid metal block of material with acoustic impedance that is similar to the acoustic impedance of silicon substrate used for a CMUT is provided. Since the solid block of material may provide high heat conductivity and a stiff mechanical support without acoustic attenuation, the block is formed to prevent reflections of acoustic energy back toward the sensor. In one embodiment, a Rayleigh dump is formed on a surface of the solid block of material away from the transducer material. Acoustically absorbing materials are provided along the surface with the Rayleigh dump. As acoustic energy propagates towards the surface, the acoustic energy is reflected at angles away from the transducer material. Some of the acoustic energy propagates through the surface into the attenuating material. After multiple reflections within the Rayleigh dump, the acoustic energy is eventually dissipated through the acoustic attenuation of the additional material alongside the surface.

### BACKGROUND

The present invention relates to acoustic absorber for ultrasound transducers. In particular, sound absorbing backings are provided for ultrasound transducers.

In medical diagnostic ultrasound imaging, acoustic energy is generated by transducer material or devices. The acoustic energy is transmitted into a patient and echoes are received in response to the transmission. The transmissions are directional, such as propagating away from a surface of the transducer material adjacent to a patient. Transducer material generates acoustic energy along an axis in both directions. To prevent longitudinal waves propagating away (i.e., a backward traveling wave) from the patient from causing clutter or undesired reflections back to the transducer, a backing block is provided. The backing block absorbs acoustic energy to prevent undesired reflections.

For piezoelectric or PZT ceramic transducer materials, the backing block also defines the acoustic impedance at the surface of the transducer material away from the patient. The acoustic impedance of the PZT ceramic typically has an acoustic impedance of 20 to 30 MRayl and the backing blocks typically have an acoustic impedance of 3 to 12 MRayl. For example, an epoxy filled with small particles is used to absorb acoustic energy without scattering or reflecting the energy. The impedance discontinuity at this surface reflects some of the backward traveling wave. To minimize this reflection, a backing material must be used which has an acoustic impedance which matches the PZT, however absorbing materials with impedances this high do not exist and are difficult to synthesize. The amplitude of this reflection is generally 7% to 19% of the amplitude of the energy generated by the transducer, and by design is incorporated into the transducer response and influences the sensitivity and bandwidth. Its deleterious impacts are mitigated by the attenuation of this component in the PZT's mechanical and electrical losses, propagation away from the transducing material as electrical energy into the electrical circuitry, and propagation away from the transducing material as acoustic energy into the patient.

In capacitive membrane ultrasound transducers (CMUT's) made from micro-machined silicon, several mechanisms contribute to undesired reflections back to the transducing element. The transducing mechanism is the electrostatic force between a membrane electrode and a substrate electrode. Opposite and equal forces act on these two electrodes. The force on the substrate electrode is associated with undesired signal. Also, as a CMUT membrane flexes to generate or receive acoustic energy, acoustic energy coupled into the supporting silicon substrate causes undesired reflections from the interface with the supporting material. As there is very little acoustic absorption in the silicon substrate, these acoustic signals must be attenuated in materials added to the device. Since silicon and other materials used for CMUT transducers has a longitudinal impedance of about 17 to 20 MRayl, backing block materials used for PZT transducers may also create a reflective interface with the substrate in CMUT's.

Appropriate materials available for use as backing blocks are limited. Additionally, many backing block materials may be selected to provide at least some heat conductivity. For manufacturing purposes, the backing block may be selected to be as stiff as possible for providing mechanical support to the assembled array. These and other considerations limit the available acoustically attenuating materials used for a backing block.

Other transducer related materials are selected for acoustic properties. Matching layers are used PZT transducers to transition acoustic impedance from the transducer material to a patient. Where a wedge or block is designed to be placed between the transducer and the patient, the wedge or block has similar acoustic impedance to the patient. To avoid reflections from a surface of a wedge not contacting the surface of the transducer or patient, a Rayliegh dump in an absorbing material may be added to that surface.

### BRIEF SUMMARY

The present invention is defined by the following claims, and nothing in this section should be taken as a limitation on those claims. By way of introduction, the preferred embodiments described below include sound absorption backings for ultrasound transducers and methods of absorbing sound. A block of material with similar acoustic impedance to the transducer material is provided adjacent to the material. For example, a solid metal block of material with acoustic impedance that is similar to the acoustic impedance of a silicon substrate used for a CMUT is provided. Since the solid block of material may provide high heat conductivity and stiff mechanical support but without acoustic attenuation, the block is formed to prevent reflections of acoustic energy back to the transducer material. In one embodiment, an anechoic surface, such as a Rayleigh dump, is formed on a surface of the solid block of material away from the transducer material. Acoustically absorbing materials are provided along the anechoic surface. As acoustic energy contacts the surface, the acoustic energy is reflected at angles away from the transducer material. With each reflection, some of the acoustic energy propagates through the surface into the attenuating material. After multiple reflections on the surface, the acoustic energy traversing the surface is eventually dissipated through the acoustic attenuation of the additional material adjacent the surface. Less, minimal or no acoustic energy propagates back to the transducer material.

In a first aspect, an ultrasound transducer for converting between an acoustic and electrical energy is provided. A backing block is provided on at least one side of transducer material. The backing block includes an anechoic surface.

In a second aspect, an ultrasound transducer for converting between acoustic and electrical energy is provided. A transducer material is formed as an array of elements. A backing block is provided on at least one side of the transducer material. The backing block includes a block of a first material adjacent to the transducer material. The first material may have substantially no acoustic attenuation at a range of frequencies for operation of the array of elements.

In a third aspect, an ultrasound transducer for converting between acoustic and electrical energy is provided. A backing block is provided on at least one side of the transducer material. The backing block includes a solid block of a first material adjacent to the transducer material. The first material has a thermal conductivity greater than the transducer material.

In a fourth aspect, a capacitive membrane ultrasound transducer is provided for converting between acoustic and electrical energy. A silicon substrate has a plurality of flexible membranes. A backing block is adjacent to the silicon substrate. The backing block has a solid block of first material adjacent to the transducer material. A block of second material is positioned adjacent to the first material away from the silicon substrate. A surface of contact between the first and second materials has at least one area angled relative to the silicon substrate to reflect acoustic energy away from the silicon substrate.

In a fifth aspect, a method for attenuating acoustic energy in a backing block is provided. Acoustic energy is transmitted into the backing block. The acoustic energy reflects off of a Rayleigh dump surface in the backing block. The acoustic energy passing through the surface is absorbed.

Further aspects and advantages of the invention are discussed below in conjunction with the preferred embodiments.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different views.

FIG. 1 is a cross-section diagram of one embodiment of an ultrasound transducer with transducer material and a backing block;

FIG. 2 is a graphical representation of acoustic reflections in one embodiment of a Rayleigh dump; and

FIG. 3 is a flowchart diagram of one embodiment of a method for attenuating acoustic energy in a backing block.

### DETAILED DESCRIPTION OF THE DRAWINGS AND PRESENTLY PREFERRED EMBODIMENTS

Available backing block materials for attenuating ultrasound energy with similar acoustic impedance to silicon for CMUT transducers are limited. For example, longitudinal wave acoustic impedance of aluminum and silicon are a good match, resulting in low acoustic reflection coefficients from the interface between the two materials. Acoustic power launched into the substrate enters the aluminum with little reflection, resulting in no or limited reflection. Aluminum provides little or no acoustic attenuation, so the acoustic energy is deposited into an absorbing material. Absorbing materials with acoustic impedance similar to aluminum and silicon may be difficult to synthesis or may be unavailable. An anechoic Rayleigh dump is formed on the surface of the aluminum spaced away from the transducer material. The Rayleigh dump acts to deposit the acoustic power into an absorber placed on the surface of the aluminum. Due to the shape of the Rayleigh dump, little or no acoustic energy is reflected back towards the transducer material even with the differences in acoustic impedance between the aluminum and the acoustically absorbing material.

While a specific embodiment is discussed above, embodiments using ceramic or piezoelectric transducer materials with different metals or non-metal backing block materials may be used. The use of an anechoic surface may allow selection of materials that have different acoustic impedances, thermal conductivities, little or no acoustic attenuation or other desired characteristics.

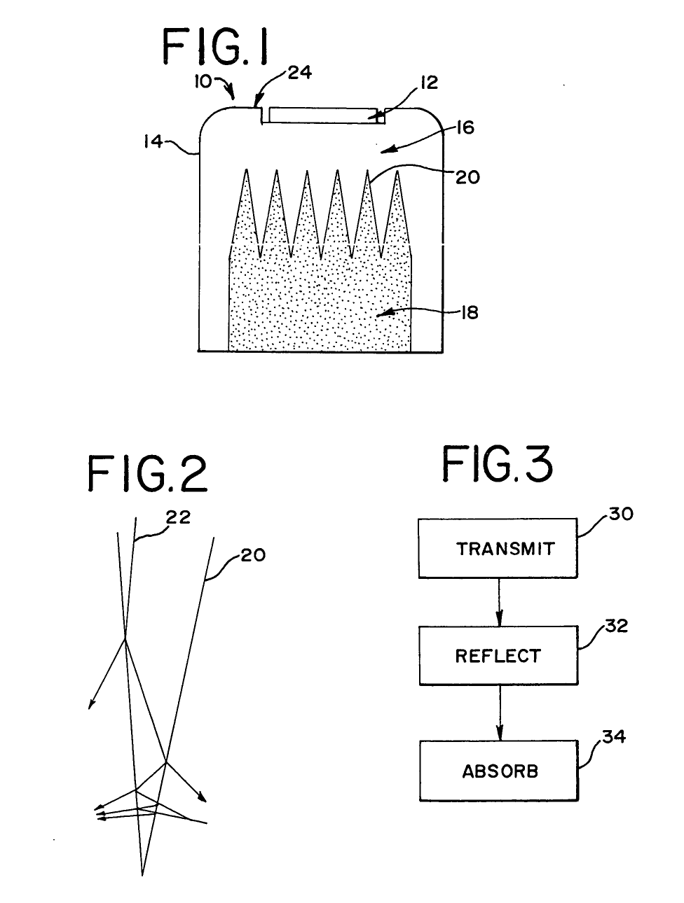

FIG. 1 shows one embodiment of a cross-section view of a transducer 10. The transducer 10 has a linear array of elements, a multi-dimensional array of elements or a single element. Any of various transducer stacking materials, including signal traces, electrodes, matching layers and/or lens may be used. The transducer 10 converts between acoustic and electrical energy. The transducer 10 is used for medical diagnostic ultrasound imaging in one embodiment, but may be used for sonar, materials testing or other ultrasound transmission and reception.

The transducer 10 shown in FIG. 1 includes transducer material 12 and a backing block 14. The transducer material 12 is piezoelectric, piezoelectric composite, silicon, other CMOS processed material, or other now known or later developed materials for converting between acoustical and electrical energies. In one embodiment, the transducer material 12 is a silicon substrate with one or more flexible membranes formed within or on the silicon substrate. The flexible membrane has an electrode on at least one surface for transducing between energies using a capacitive effect, such as provided in capacitive membrane ultrasound transducers. The membrane is formed with silicon or other materials deposited or formed on the silicon substrate.

As shown in FIG. 1, the transducer material 12 corresponds to a cross-section of a single element in a linear array. The remaining elements of the array extend along the backing block 14 perpendicular to the plane of FIG. 1. In alternative embodiments, the transducer material 12 shown comprises a linear array extending from the left side to the right side of FIG. 1 with the full extent of the array shown. The backing block 14 in either of the array embodiments is positioned adjacent to the silicon substrate and extends along at least one, two, all or a subset of the elements of the array.

The backing block 14 includes two materials 16 and 18 with an anechoic surface 20. The backing block 14 is positioned adjacent to the transducer material 12 to prevent undesired signals propagating through the backing block 14 from reflecting back to the transducer material 12. The anechoic surface 20 is a Rayleigh dump in one embodiment, but other now known or later developed anechoic surfaces may be used. The surface 20 is spaced away from the transducer material 12 and includes one or more peaks in cross-section. For example, the surface 20 forms a plurality of pyramids in three-dimensional space. As another example, the surface 20 forms a plurality of parallel ridges extending in parallel width, perpendicular to or at an angle relative to the direction of the array of elements. As shown in cross-section of FIG. 1, the pyramids or ridges provide a plurality of peaks in cross-section. Due to the shape of the surface 20, at least one area of the surface 20 is angled relative to the transducer material 12 to reflect acoustic energy away from the transducer material 12. The angle between the faces of the surface 20 is about 20 degrees in one embodiment, but may be greater or lesser. While all the peaks of the surface 20 are shown as a same height, the peaks or valleys may vary or be different along one or more dimensions. The width between the peaks is larger than a single wavelength. While shown as having six peaks and an associated five valleys of the second material 18 or five peaks with six valleys of the first material 16, any number of peaks and valleys may be provided for the surface 20 including a single peak or valley. The distance between the peaks or valleys is at least five wavelengths in one embodiment, but a lesser or varying distance may be used. For a typical one-dimensional medical imaging array, about five wavelengths distance may translates to about five, six or fewer peaks or valleys. While shown as having peaks and associated valleys running parallel, the distance between the peaks and valleys may vary along their length.

FIG. 2 shows incident acoustic energy 22 into a valley or dump formed in the surface 20. As the incident energy contacts the surface 26, some of the energy reflects at an angle from the surface while some energy is passed through the surface. Following the first reflection, multiple reflections are repeated at decreasing angles. The decreasing angles approach an angle perpendicular to the two side walls of the surface 20, avoiding or minimizing reflections back towards the transducer material 12. After a number of reflections, the angle incident reaches 90 degrees and the acoustic wave begins to be reflected back towards the transducer material 12. Since the acoustic energy loses power with each reflection, minimal energy is reflected back to the ultrasound transducer 12.

Due to the characteristics of the second material 18 used to form the surface 20, the acoustic energy passing through the surface 20 is attenuated or absorbed. The Rayleigh dump or surface 20 is formed at an interface between the two different materials 16, 18, but may be formed spaced from one or both of the two materials by a third material.

The first material 16 is any of various now known or later developed materials with an acoustic impedance matched to the acoustic impedance of the transducer material 12. Matched acoustic impedance includes acoustic impedance within 10% or a same acoustic impedance between the material 16 and the transducer material 12, but a greater difference may be provided. For example, where the transducer material 12 is silicon or a CMUT transducer, the material 16 is a metal material, such as a solid block of aluminum or other metal. Solid is material with a consistent molecular make-up, such as without filler particles. Silicon substrate has a longitudinal acoustic impedance of about 17 to 20 MRayl. Aluminum has a longitudinal wave acoustic impedance of about 17 MRayl. Other materials with matched or similar longitudinal wave acoustic impedances to silicon include Bearing Babbitt (23.2 MRayl), tin (24.2 MRayl), lead (24.6 MRayl), indium (18.7 MRayl), solder, silicon, beryllium (24.1 MRayl), cadmium (24.0 MRayl), flint glass (16.0 MRayl), Macor (14.0 MRayl), lead Metaniobate (20.5 MRayl), liquid sodium (21.32 MRayl), granite (17.6 MRayl) and Bismuth (21.5 MRayl). Other materials for matching to a silicon substrate or for matching to other transducer materials may be used. Similar or the same materials may have different acoustic impedance values.

In one embodiment, the material 16 is a solid block of material, such as a solid block of metal or metal alloy. In other embodiments, additional materials are formed within or as part of the material 16, such as providing fluid cooling channels, pockets of filler material or other particles. As an alternative, the material 16 is a liquid material enclosed within a housing with similar acoustic impedance. Where cooling channels or liquid coolants are provided in the material 16, liquids with similar acoustic impedances are used to avoid reflections.

Different ones of first materials may be used in different situations or for different reasons. For example, granite and other metallic materials have thermal conductivities greater than the transducer material 12. Since thermal considerations may be important for ultrasound applications, a higher thermal conductivity may be desired. In addition to having a higher thermal conductivity, the temperature coefficient match may be an important consideration in order to avoid distortion of a transducer due to internal thermal gradients. Rigidity, stiffness or mechanical support may be important for forming the transducer 10. The material 16 acts to support the back of the transducer material 12. Various materials, such as a solid block of aluminum, granite, flint glass, Macor and tin, may provide non-brittle, durable materials for supporting the manufacturer and use of the array 10.

Many of the materials discussed above for adjacent to the transducer material 12 provide no, minimal or limited acoustic attenuation at a range of frequencies of operation of the array of elements. For example, a solid block of aluminum material 16 provides no or little acoustic attenuation at 1 to 12 MHz frequency range. To absorb the acoustic energy and avoid reflections back to the transducer material from the backing block 14, the second material 18 forming the anechoic surface 20 is an acoustic attenuative material.

The second material 18 is any of now known or later developed materials for attenuating acoustic energy, such as ultrasound acoustic energy. For example, a cured epoxy with or without filler material is used. Where filler material is provided, the filler material is small enough to avoid reflections of acoustic energy. The second material 18 has an acoustic impedance that is at least 30 percent less than the acoustic impedance of the transducer material 12 in one embodiment. In alternative embodiments, a lesser difference in acoustic impedance is provided. In one embodiment the second material 18 is selected to have as high a longitudinal wave acoustic impedance as possible while still attenuating the ultrasound energy. For example, filler material is added to synthesize an acoustic impedance of about 12 MRayl or more. Materials with any acoustic impedance may be used, such as materials with a range of 3 to 12 MRayl. Higher or lower impedance may be provided. Where the first material 16 provides a rigid structure, the second material 18 is selected for desired attenuation properties with minimal or no consideration of rigidity. For example, acoustically absorbing gels, foams, epoxies, liquids, or other materials with excellent, no or some mechanical support are used. The second material 18 may have a lesser thermal conductivity than the first material 18 since the first material 16 acts to cool the transducer 10, allowing the second material 18 to be selected for acoustic properties rather than thermal conduction properties. Combinations of different materials may also be provided in a mixed or structural combination on a micro or macro level.

The second material 18 is spaced from the transducer material 12 by the first material 16. The acoustically attenuative material 18 is positioned at the surface 20 adjacent to the block of the first material 16. In alternative embodiments, the second acoustically attenuative material 18 is spaced from the surface 20 by one or more other materials which, if they are made of a material with an impedance intermediate between the impedances of materials 16 and 18, may function as a quarter-wave matching layer for the surface 20. The Rayleigh dump or anechoic surface 20 passes ultrasound energy into the acoustically attenuative material 18. Since the material 18 has a greater acoustic absorption than metal or other material 16 adjacent to the transducer material 12, the acoustic energy is primarily attenuated by the second material 18.

The backing block 14 and the first material 16 are shaped in cross section as shown in FIG. 1, but other shapes may be used. A lip or edge 24 is provided on one or both sides of the transducer material 12. The edge 24 supports signal traces that contact an upper surface of the transducer material 12. Where signal traces are formed along the lower surface of the transducer material 12, the backing block 14 includes the lip 24 or is flat without the lip 24. For supporting the second material 18 within the backing block 14, the first material 16 extends downward to house the second material 18 on the sides. In alternative embodiments, the second material 18 extends all the way to the sides of the backing block 14 without the housing of the first material 16.

The backing block 14 is manufactured by forming the first material 16 in a desired shape and then forming the second material 18 on or within the first material 16. For example, the first material 16 is an extruded metal block, wire cut at the ends with or without an additional end cap formed on the material 16. Any of various extrusion, molding, cutting or other now known or latter developed processes for forming the first material 16 under the desired shape may be used. The second material 18 is then molded in, deposited in, or cured in the first material 16. For example, an epoxy with or without filler is poured within the first material 16 and allowed to cure. Alternatively, the second material 18 is formed using extrusion, molding, thermoplastic injection or cutting processes to mate with the first material 16. The second material 18 bonds to the first material 16. Alternatively, an additional bonding agent is provided along the surface 20. In yet another embodiment, a bonded or attached plate is positioned over the first material 16 and second material 18 to maintain the second material 18 adjacent to the surface 20 and the first material 16, as might be used to contain an attenuating fluid or freely flowing plastic material. The transducer material 12 is then stacked, bonded or otherwise formed adjacent to or on the backing block 14.

FIG. 3 shows one embodiment of a method for attenuating acoustic energy in a backing block. The transducer of FIG. 1 or another transducer is used to implement this method. Additional, different or fewer acts may be provided.

In act 30, acoustic energy is transmitted into a backing block. Acoustic energy is generated from a transducer material as a longitudinal or other wave modes extending as a wave from the transducer material. Energy extending towards a patient or surface to be sensed is desired, but energy extending in the opposite direction is undesired. For example, a membrane of a capacitive membrane ultrasound transducer flexes in response to electrical signals. The flexing generates acoustical energy that is transmitted perpendicularly in both directions away from the membrane. The backing block absorbs the acoustic energy transmitted in the undesired direction to avoid echoes.

In act 32, the acoustic energy transmitted into the backing block is reflected off of an anechoic dump surface in the backing block. Due to the angle of the anechoic surface, the reflected wave is initially scattered away from the sensor. After a number of reflections, the angle incidence of the waveform reaches 90 degrees. The acoustic energy that remains is then reflected multiple times within the anechoic surface or Rayleigh dump, eventually being reflected back towards the sensor with no or a greatly reduced power.

In act 34, the acoustic energy passing through the surface into the material 18 is converted to heat as it propagates through the material 18 by the losses of the material. At each reflection, some of the acoustic power or energy passes through the surface rather than being reflected off of the surface. As each reflection occurs, more or additional acoustic power is passed through the surface and absorbed by an acoustically attenuative material.

In another embodiment, the first material 16, such as an aluminum member, is formed into a structural frame for the entire transducer such that there are no surfaces behind the transducer material 12 that reflect back toward the transducer material 12. The first material 16 acts as a wave guide. The acoustic absorber is located remotely from the transducer material 12. The waveguide may be coated with acoustic absorbent material along the length of the waveguide. An acoustic dump is provided at the terminus. Heat generated by the absorption of the acoustic energy is away from the transducer material 12. The heat generation from absorption is removed from proximity to the patient.

While the invention has been described above by reference to various embodiments, it should be understood that many changes and modifications can be made without departing from the scope of the invention. For example, piezoelectric or composite ceramic transducer materials may be used with any of various backing block materials. Even though a backing block material may attenuate acoustic energy, a Rayleigh dump or anechoic surface may assist in acoustic absorption or allow smaller backing blocks.

It is therefore intended that the foregoing detailed description be regarded as illustrative rather than limiting, and that it be understood that it is the following claims, including all equivalents, that are intended to define the spirit and scope of this invention.

1. An ultrasound transducer for converting between acoustic and electrical energy, the transducer comprising:
transducer material;
a backing block on at least one side of the transducer material, the backing block including an anechoic surface.

2. The transducer of claim 1 wherein the transducer material comprises a capacitive membrane connected with silicon, the backing block adjacent the silicon.

3. The transducer of claim 1 wherein the transducer material comprises an array of elements, the array of elements adjacent the backing block.

4. The transducer of claim 1 wherein the anechoic surface comprises a Rayleigh dump with a surface having one of: at least one peak, at least one valley and combinations thereof in cross-section.

5. The transducer of claim 1 wherein the backing block comprises first and second different materials, the anechoic surface being at an interface of the first material with the second material.

6. The transducer of claim 5 wherein the first material is adjacent to the transducer material and the second material is spaced from the transducer material by the first material, the first material having an acoustic impedance within 10% of an acoustic impedance of the transducer material, the second material having an acoustic impedance at least 30% less than the acoustic impedance of the transducer material.

7. The transducer of claim 1 wherein the backing block comprises a block of material having acoustic impedance within 10% of an acoustic impedance of the transducer material.

8. The transducer of claim 7 wherein the block of material comprises a metal material.

9. The transducer of claim 8 wherein the metal material comprises one of: Aluminum and an Aluminum alloy.

10. An ultrasound transducer for converting between acoustic and electrical energy, the transducer comprising:
transducer material having an array of elements;
a backing block on at least one side of the transducer material, the backing block including a block of first material adjacent to the transducer material, the first material having substantially no acoustic attenuation at a range of frequencies for operation of the array of elements.

11. The transducer of claim 10 wherein the first material comprises metal.

12. The transducer of claim 11 wherein the first material comprises Aluminum.

13. The transducer of claim 10 wherein the block of first material has a surface with a Rayleigh dump.

14. The transducer of claim 13 wherein the backing block further comprises an acoustically attenuative second material positioned at the Rayleigh dump adjacent to the block of first material.

15. The transducer of claim 10 wherein the first material has a thermal conductivity greater than the transducer material.

16. The transducer of claim 10 wherein the transducer material comprises silicon.

17. An ultrasound transducer for converting between acoustic and electrical energy, the transducer comprising:
transducer material;
a backing block on at least one side of the transducer material, the backing block including a solid block of first material adjacent to the transducer material, the first material having a thermal conductivity greater than the transducer material.

18. The transducer of claim 17 wherein the solid block of material comprises a solid metal.

19. The transducer of claim 17 wherein the solid block of material has a surface spaced away from the transducer material with a Rayleigh dump, a second material with a lesser thermal conductivity than the first material positioned adjacent to the Rayleigh dump.

20. The transducer of claim 17 wherein the first material has an acoustic impedance within 25% of an acoustic impedance of the transducer material.

21. A capacitive membrane ultrasound transducer for converting between acoustic and electrical energy, the transducer comprising:
a silicon substrate supporting a plurality of flexible membranes;
a backing block adjacent the silicon substrate, the backing block having a solid block of first material adjacent to the transducer material, a block of second material positioned adjacent to the first material away from the silicon substrate wherein a surface of contact between the first and second materials has at least one area angled relative to the silicon substrate to reflect acoustic energy away from the silicon substrate.

22. The transducer of claim 21 wherein the surface of contact forms a Rayleigh dump.

23. The transducer of claim 21 wherein the solid block of first material comprises a metal material, the second material having a greater acoustic absorption than the metal material.

24. A method for attenuating acoustic energy in a backing block, the method comprising:
(a) transmitting acoustic energy into the backing block;
(b) reflecting the acoustic energy off of a Rayliegh dump surface in the backing block; and
(c) absorbing the acoustic energy passing through the surface.

25. The method of claim 24 wherein (b) and (c) comprises providing the surface between a solid block of a first material and a second material, the second material having a greater acoustic attenuation than the first material.

26. The method of claim 24 wherein (a) comprises transmitting with a membrane of a capacitive membrane ultrasound transducer.

27. The transducer of claim 1 wherein the backing block comprises a wave guide.
